(19) United States
(12) Patent Application Publication
(10) Pub. No.: US 2006/0204989 A1
(43) Pub. Date: Sep. 14, 2006

(54) Comparative analysis of extracellular RNA species

(76) Inventor: Michael S. Kopreski, Long Valley, NJ (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/357,399

(22) Filed: Feb. 17, 2006

Related U.S. Application Data
(63) Continuation-in-part of application No. 10/658,873, filed on Sep. 5, 2003.
Application No. 10/658,873 is a continuation-in-part of application No. 10/013,868, filed on Oct. 30, 2001, now Pat. No. 6,939,671.
Application No. 10/013,868 is a continuation of application No. 09/155,152, filed on Sep. 22, 1998, now Pat. No. 6,329,179.

(51) Int. Cl. C12Q 1/68; C12P 19/34

(52) U.S. Cl. 435/6; 435/91.2

(57) ABSTRACT

The invention provides methods and kits for enabling quantitative or qualitative analysis of extracellular RNA species in non-cellular bodily fluids including plasma and serum to detect, infer, evaluate, or monitor cancer and other neoplasia or other diseases of interest.

This application is a continuation-in-part of U.S. Ser. No. 10/658,873, filed Sep. 5, 2003, which is a continuation-in-part of U.S. Ser. No. 10/013,868, filed Oct. 30, 2001, now U.S. Pat. No. 6,939,671, which is a continuation of U.S. patent application Ser. No. 09/155,152, filed Sep. 22, 1998, now U.S. Pat. No. 6,329,171 B1, the disclosures of each of which are explicitly incorporated by reference herein.

### BACKGROUND OF THE INVENTION

Ribonucleic acid (RNA) plays an essential role in the translation of the genetic code to produce proteins necessary for cellular function, both in normal cells and neoplastic or diseased cells. In particular, RNA including transfer RNA, messenger RNA or messenger-like RNA, and ribosomal RNA carry and translate the genetic code to sites of protein production. Further, double-stranded RNA and species therefrom, including small inhibitory RNA (short interfering RNA or siRNA), and micro RNA or miRNA, play an important role in silencing genetic expression. Other RNA species are found within ribonucleoproteins. For example, telomerase RNA is a critical component of telomerase, an important ribonucleoprotein over-expressed in most cancers. The pathogenesis and regulation of cancer is thus dependent upon RNA-mediated translation and/or inhibitory control of specific genetic code, said genetic code often reflecting mutational events or other alterations within deoxyribonucleic acid (DNA), including epigentic alterations such as hypermethylation, microsatellite alterations, loss of heterozygosity, translocations, deletions, and point mutations. Further, other RNA species and their associated proteins, although not necessarily being directly involved in neoplastic pathogenesis or regulation, may provide recognizable characterization of neoplasia or other disease by being inappropriately expressed or over-expresessed or elevated. Such overexpression of RNA thus can delineate cancer or other diseases, including but not limited to cardiovascular diseases and neurological diseases. Recognition of the presence or overexpression of specific RNA, including both coding and non-coding RNA, can enable identification, detection, inference, monitoring, or evaluation of any neoplasm, whether benign, malignant, or premalignant, in humans and animals, or of other diseases in the human or animal.

U.S. Pat. No. 6,329,179 B 1, incorporated herein in its entirety, teaches that both tumor-associated and non-tumor associated mammalian RNA are detectable in plasma and serum. Total RNA is intended herein to refer to a mixture or collection of heterogeneous RNA species or fragments thereof, and is to be understood in the cancer patient to comprise both tumor-associated and non-tumor-associated RNA and further it will be understood that it is inclusive of heterogeneous RNA of various RNA species and types, which can include messenger RNA, ribosomal RNA, transfer RNA, micro RNA, short interfering RNA, and mitochondrial RNA, and specific species thereof. RNA comprising heterogeneous RNA, for example total RNA, can be extracted from plasma or serum or other non-cellular bodily fluid fractions, the RNA of interest or its cDNA is amplified qualitatively or quantitatively, and the amplified product of an RNA or cDNA species of interest detected. Subsequent art supports these teachings by demonstrating that extracellular RNA of various RNA species are detectable in bodily fluids, for example in co-owned U.S. Pat. No. 6,607,898; Kopreski et al., 1999, Clin. Cancer Res. 5: 1961-1965; Dasi et al., 2001, Lab. Investigation 81: 767-769; Hasselmann et al., 2001, Oncol. Rep. 8: 115-118; Ng et al., 2002, Clin. Chem. 48: 1212-1217; Chen et al., 2000, Clin. Cancer Res. 6: 3823-3826; Silva et al., 2001, Clin. Cancer Res. 7: 2821-2825; Silva et al., 2001, Oncol. Rep. 8: 693-696; Gal et al., 2001, Ann. NY Acad. Sci. 945: 192-194; Durie et al., 2000, Acta Oncol. 39: 789-796; Fleischhacker et al., 2001, Ann. NY Acad. Sci. 945: 179-188; Miura et al., 2003, Oncology 64: 430-434; Kopreski et al., 2001, Ann. NY Acad. Sci. 945: 172-178; and Wong et al., J. Clin. Pathol. 2004, 57: 766-768, said references incorporated herein in their entirety. Detection of tumor-associated RNA in plasma or serum or non-cellular bodily fluid fractions thus provides a method for detecting, diagnosing, inferring, evaluation, characterization, or monitoring cancer or premalignancy in a human or animal. Similarly, detection of other disease-related RNA in plasma, serum, or non-cellular bodily fluids enable the detection, diagnosis, evaluation, characterization, or monitoring of other diseases of interest.

Neoplasia is characterized by varying degrees of invasiveness, metastatic potential, and resistance or responsiveness to particular therapies. Furthermore, these characteristics for a given neoplasia may change over time, for example by becoming progressively more malignant, invasive, metastatic, heterogeneous, undifferentiated, or treatment-resistant. Phenotypic changes often reflect underlying molecular changes. In particular, the relative or absolute amounts or ratio of particular RNA species, including coding and non-coding species, to each other, and/or to DNA, and/or to proteins can determine the characteristics of the neoplasia, and further enable the diagnosis, detection, evaluation, or monitoring of cancer and premalignancy. Similarly, the relative or absolute amounts or ratio of particular RNA species, including coding and non-coding species, to each other, and/or to DNA, and/or to proteins can determine characteristics of non-neoplastic diseases and conditions, including but not limited to cardiovascular diseases, neurological diseases, endocrinological diseases, respiratory conditions, gastrointestinal diseases, and obstetrical states and conditions, and furthermore enable the diagnosis, detection, evaluation, or monitoring of said disease or condition.

Analysis in an absolute or relative fashion of extracellular RNA species to each other, and/or to extracellular DNA, and/or to extracellular protein, would thus be useful as a method for detecting, diagnosing, inferring, evaluating, characterizing, or monitoring cancer or premalignancy or other diseases or conditions in a human or animal. Said analysis would further enable selection and monitoring of treatment. Although extracellular RNA is generally protected from the degrading effects of plasma RNase by being protected within encapsulated particles such as apoptotic bodies, a variable disruption of the particles or apoptotic bodies may occur over time or consequent to mechanical manipulations such as freeze-thawing or centrifugation, leading to variable rates of degradation of the extracellular RNA through its exposure to nucleases. To optimize the reliability and reproducibility of such analysis, there is a need in the art for methods that maintain the stability of extracellular RNA species and their concentrations and relative ratios within a plasma or serum or non-cellular bodily fluid specimen over time.

Thus, there is a need in the art for methods of analyzing the amount or concentration or relative ratio of two or more plasma or serum or non-cellular (acellular) bodily fluid RNA species or fragments thereof to permit diagnosis, detection, inference, evaluation, or monitoring of disease including neoplastic disease or other disease of interest in a human or animal.

Furthermore, there is a need in the art for methods of analyzing in quantitative or qualitative fashion the amount or concentration or ratio of one or more extracellular RNA species to the amount or concentration of total RNA or extracellular DNA or protein present in the plasma, serum, or non-cellular bodily fluid of a human or animal for the diagnosing, detecting, inferring, evaluating, or monitoring cancer and other diseases or conditions in the human or animal. Furthermore, there is a need for methods of analyzing in quantitative or qualitative fashion the amount or concentration or ratio of one or more extracellular RNA species to a reference value or amount or concentration or ratio or specimen.

### SUMMARY OF THE INVENTION

This invention provides methods for diagnosing, detecting, inferring, evaluating, or monitoring cancer or other diseases or conditions in a human or animal by determining the amount, concentration, ratio, or other quantitative or qualitative assessment between two or more extracellular RNA species in plasma or serum or other bodily fluid from a human or animal, where in a preferred embodiment the extracellular RNA species have been stabilized from degradation caused by nucleases. The present invention thus provides methods to enhance the intra-specimen stability and reproducibility of the ratio between two or more RNA species in a plasma or serum or bodily fluid specimen, and kits thereof.

The invention further provides methods for analyzing one or more specific extracellular RNA species in plasma or serum or bodily fluid to another within said specimen, or to extracellular total RNA, extracellular DNA, or extracellular protein within said plasma, serum, or bodily fluid specimen. The methods provided by the invention comprise qualitative or quantitative determination of the amount or concentration or ratio between at least two RNA species in a non-cellular (acellular) bodily fluid specimen by any of means known to the art, including but not limited to nucleic acid amplification, signal amplification, spectroscopy including mass spectroscopy, and hybridization methods using detectably-labeled probes. The methods provided by the invention further comprise qualitative or quantitative determination of a least one extracellular RNA species within a bodily fluid specimen to one or more of the following group within said specimen: total extracellular RNA, total extracellular DNA, one or more extracellular DNA species, one or more extracellular proteins. It is to be understood that within this specification, RNA species refers to an RNA species selected from one or more of messenger RNA (mRNA), ribosomal RNA (rRNA), transfer RNA (tRNA), regulatory or inhibitory (interfering) RNA such as short interfering RNA (siRNA) and micro RNA (miRNA), mitochondrial RNA (mtRNA), coding RNA, non-coding RNA, RNA having a sequence complimentary to a mutated or altered DNA, RNA having a sequence complimentary to non-mutated DNA, mRNA splice variants, and ribonucleoprotein RNA.

According to a first aspect of the present invention, there is provided methods for detecting, diagnosing, inferring, evaluating or monitoring disease, particularly cancer or neoplastic disease in a human or animal, the method comprising the steps of isolating a predominantly non-cellular or acellular fraction of a bodily fluid obtained from a human or animal, wherein the non-cellular fraction may be plasma, serum, or other non-cellular (acellular) bodily fluid; combining the non-cellular fraction of the bodily fluid with an agent that protects or stabilizes RNA from degradation by RNase (hereafter referred to as stabilizing agent or stabilizer agent); thereafter extracting RNA from the non-cellular fraction of the bodily fluid, such as from plasma, serum or other non-cellular bodily fluid specimen of a human or animal, wherein the extracted RNA may comprise total RNA or a heterogeneous mixture of RNA species or specific RNA species, determining quantitatively or qualitatively the amount or concentration of at least two RNA species from a fraction of said plasma, serum or other non-cellular bodily fluid fraction, wherein quantitative or qualitative determination of the amount or concentration of said RNA species thereby detects, diagnoses, infers, or monitors or enables evaluation of a cancer or neoplastic disease or other disease or condition in a human or animal. In a particularly preferred embodiment, at least two of the RNA species are tumor-associated RNA, and cancer or neoplastic disease is detected, diagnosed or inferred or evaluated when the relative or absolute amount or concentration of at least one RNA species from the plasma, serum or non-cellular bodily fluid fraction from a human or animal is greater than the amount or concentration of another RNA species from the plasma, serum, or non-cellular bodily fluid fraction from said human or animal. In another particularly preferred embodiment, at least two of the RNA species are tumor-associated RNA and a third RNA species is not cancer-specific, and cancer or neoplastic disease is detected, diagnosed or inferred or evaluated when the relative or absolute amount or concentration of a least two of the tumor-associated RNA species from the plasma, serum, or non-cellular bodily fluid fraction of a human or animal is greater than the amount or concentration of the non-tumor specific RNA species in the plasma, serum, or non-cellular bodily fluid fraction from said human or animal. In another preferred embodiment, at least two of the RNA species are disease-specific, and the disease is diagnosed, detected, inferred, evaluated, characterized, or monitored in a human or animal when the relative or absolute amount or concentration of at least one RNA species from the plasma, serum, or non-cellular bodily fluid fraction is greater than the amount or concentration of another RNA species from the plasma, serum, or non-cellular bodily fluid fraction from said human or animal. In another preferred embodiment, at least two of the RNA species are disease-specific RNA and a third RNA species is not disease-specific, and a disease is detected, diagnosed or inferred or evaluated when the relative or absolute amount or concentration of a least two of the disease-specific RNA species from the plasma, serum, or non-cellular bodily fluid fraction of a human or animal is greater than the amount or concentration of the non-disease specific RNA species in the plasma, serum, or non-cellular bodily fluid fraction from said human or animal.

In another preferred embodiment, a cancer or neoplastic disease or other disease or condition is detected, diagnosed or inferred or evaluated when at least two of the RNA species are detected in the plasma, serum, or a non-cellular bodily fluid fraction of a human or animal.

In a preferred embodiment of the invention, the stabilizing agent is a RNase inhibitor or a RNase inactivator which stabilizes extracellular RNA in plasma, serum, or a non-cellular bodily fluid fraction. In another preferred embodiment, the stabilizing agent is a RNase inhibitor or RNase inactivator that stabilizes total extracellular RNA inclusive of non-tumor RNA prior to its being isolated from a cellular bodily fluid such as whole blood, wherein the stabilizing agent is mixed with a cellular bodily fluid.

In another preferred embodiment of the invention, the stabilizer agent hybridizes to the RNA species, whereby it stabilizes extracellular RNA in plasma, serum, or a non-cellular bodily fluid fraction. In another preferred embodiment, the stabilizing agent hybridizes to specific extracellular RNA inclusive of non-tumor RNA prior to its being isolated from a cellular bodily fluid such as whole blood, wherein the stabilizing agent is mixed with a cellular bodily fluid.

In another preferred embodiment of the invention, the stabilizer agent is a protein that binds to the RNA species in a manner that stabilizes the RNA species against degradation by RNase, whereby it stabilizes extracellular RNA in plasma, serum, or a non-cellular bodily fluid fraction. In another preferred embodiment, the stabilizing agent is a protein that binds to extracellular RNA inclusive of non-tumor RNA prior to its being isolated from a cellular bodily fluid such as whole blood, wherein the stabilizing agent is mixed with a cellular bodily fluid.

In another preferred embodiment of the invention, the stabilizer agent is one that stabilizes a phospholipid membrane, thereby stabilizing the particle or apoptotic body encapsulating extracellular RNA in plasma, serum, or a non-cellular bodily fluid fraction. In another aspect of this embodiment, the stabilizing agent is mixed directly with a cellular bodily fluid such as whole blood.

In another preferred embodiment of the invention, the stabilizer agent is one that promotes aggregation of particles or apoptotic bodies encapsulating extracellular RNA in plasma, serum, or another non-cellular bodily fluid fraction, or to extracellular RNA in a cellular bodily fluid such as whole blood, thereby protecting it from RNase. In one aspect of this embodiment the stabilizing agent is a coated bead or particle. In another aspect of this embodiment, the stabilizing agent is coated solid surface. The particle may further be a metal particle that facilitates magnetic isolation or extraction of the particle.

According to another aspect of the present invention, there is provided methods for detecting, diagnosing, inferring, evaluating or monitoring disease, including cancer or neoplastic disease in a human or animal, the method comprising the steps of isolating a predominantly non-cellular fraction of a bodily fluid obtained from a human or animal, wherein the non-cellular fraction may be plasma, serum, or other non-cellular bodily fluid; combining the non-cellular fraction of the bodily fluid with a stabilizing agent; extracting RNA from the non-cellular fraction of the bodily fluid, such as from plasma or serum or other non-cellular bodily fluid specimen of the human or animal (test specimen); determining quantitatively or qualitatively the amount or concentration of one or a plurality of RNA species from a fraction of said test specimen; and determining in an absolute or relative quantitative or qualitative fashion said amount or concentration of one or a plurality of extracellular RNA species obtained from the fraction of said test specimen against the quantitative or relative amount or concentration of one or a plurality of corresponding RNA species in reference group or specimen. In one aspect, determination of the quantitative or qualitative amounts or concentrations of RNA species with respect to each other or with respect to a reference group or value or set of values or specimen is made on the basis of a statistical analysis. In another aspect said determination against the reference group or specimen is provided by either a numerical or positive/negative assessment of each extracellular RNA species within the test specimen to the reference specimen by setting the reference group or specimen or value as the baseline standard. In one aspect of this embodiment, the numerical or positive/negative values assigned to each RNA species from the test specimen using the reference standard, to values of other RNA species within the test specimen is made, whereby an RNA expression profile or a qualitative or quantitative set of RNA patterns or a RNA signature is determined, or whereby statistical analysis is performed, that thereby detects, diagnoses, infers, or monitors a cancer or neoplastic disease or disease in a human or animal. In preferred embodiments, the reference group is a human or human population of individuals without cancer or the disease of interest. In alternative preferred embodiments, the reference group is a human or human population of individuals with cancer or the disease of interest.

According to another aspect of the present invention, there are provided methods for detecting, diagnosing, inferring, or monitoring cancer or neoplastic disease or other disease in a human or animal, the method comprising the steps of obtaining a plasma or serum or non-cellular bodily fluid from the human or animal; combining the plasma or serum specimen or non-cellular bodily fluid with a stabilizing agent; determining the amount or concentration of total extracellular RNA or of one or more RNA species from a portion of the plasma or serum or non-cellular bodily fluid; evaluating or analyzing in a quantitative or qualitative fashion said amount or concentration to that of a reference group or specimen, wherein said analysis thereby detects, diagnoses, infers, or monitors a cancer or neoplastic disease or other disease in a human or animal when the quantitative or qualitative amount or concentration of total extracellular RNA or one or more RNA species from said plasma, serum, or non-cellular bodily fluid is either greater or lesser to that demonstrated in a reference group or specimen not having a cancer, neoplastic disease, or other disease of interest, or within the expected range of a reference group or specimen with a cancer, neoplastic disease, or other disease of interest.

In preferred embodiments, the reference group is a human or human population of individuals without cancer, and cancer or neoplastic disease is detected, diagnosed or inferred when the amount or concentration of total extracellular RNA in the fraction of a non-cellular bodily fluid from a human or animal is greater than the amount or concentration of total extracellular RNA found in the reference group, or when one or more RNA species in the fraction of non-cellular bodily fluid from a human or animal is greater than the amount or concentration of said species found in the reference group. In alternative preferred embodiments, the reference group is a human or human population of individuals with cancer, and cancer or neoplastic disease is detected, diagnosed or inferred when the amount or concentration of total extracellular RNA or one or more RNA species in the fraction of a non-cellular bodily fluid from a human or animal is not significantly less than the amount or concentration of total extracellular RNA found in the reference group. The reference group may be further subgrouped an analyzed according to age, gender, race, or particular prognostic characteristics. In an alternative preferred embodiment, the amount or concentration of one or more RNA species from a non-cellular bodily fluid from a human or animal may be less than that of the reference group, whereby cancer or neoplastic disease is thereby detected, diagnosed, inferred, evaluated, or characterized. In one aspect of this embodiment, an extracellular messenger RNA is less than that of a reference group and an extracellular inhibitory or interferring RNA is greater than that of a reference group.

It is to be recognized within the scope of this invention that the amounts, concentrations, values, or ratios of one or more RNA species, DNA species, proteins, total extracellular RNA levels, or total extracellular DNA levels for a reference group or specimen may be pre-defined, and standard values or baseline values provided or utilized within the scope of the embodiments of the invention. Further, pre-defined standard values or baseline values may include a reference range to determine normal or abnormal values, and may include standards of deviation, confidence level determinations, and adjusted or pre-defined ranges or values based upon age, sex, race, or other specific parameters. Further, there may be provided a control specimen, including a control bodily fluid or control RNA, DNA, or protein, or control synthetic construct, that enables re-calibration of the pre-defined reference values or baselines based upon testing.

It is to be recognized as a particularly preferred embodiment, and within the scope of the invention, that qualitative or quantitative analysis of multiple RNA species from plasma, serum, or other non-cellular bodily fluid fraction, enables determination of a RNA pattern or RNA expression profile or RNA signature, wherein said pattern, profile, or signature thereby enables the diagnosis, detection, evaluation, characterization, or monitoring of a cancer, neoplastic disease, or other disease. In one aspect, said RNA pattern, RNA expression profile or RNA signature is analyzed visually, statistically, or mathematically, against a known or standard RNA pattern, expression profile, or signature, of a reference group or specimen with or without cancer, neoplastic disease, or other disease of interest. It is further to be recognized that the reference group or specimen(s) RNA pattern, expression profile, or signature, may be specific to a particular cancer or be intended to encompass various cancer types.

In another preferred embodiment, the invention provides methods for detecting, diagnosing, inferring, characterizing, evaluating, or monitoring cancer or neoplastic disease or other disease in a human or animal, the method comprising the steps of obtaining a plasma or serum specimen or a non-cellular bodily fluid fraction from a human or animal; combining the plasma or serum or non-cellular bodily fluid fraction with a stabilizing agent; determining directly without prior extraction on a portion of said specimen the amount or concentration of one or a plurality of extracellular RNA species in a portion of the plasma or serum or non-cellular bodily fluid; quantitatively or qualitatively determining said amount or concentration relative to that of a reference group or specimen, whereby the amount or concentration of RNA species relative to the reference group or specimen detects, diagnoses, infers, or monitors a cancer or neoplastic disease or other disease in a human or animal. In preferred embodiments, the reference group is a human or human population of individuals without cancer, and cancer or neoplastic disease is detected, diagnosed or inferred when the amount or concentration of one or a plurality of extracellular RNA species in the plasma, serum, or non-cellular bodily fluid is greater than the amount or concentration of one or a plurality of extracellular RNA species found in the reference group. In alternative preferred embodiments, the reference group is a human or human population of individuals with cancer, and cancer or neoplastic disease is detected, diagnosed or inferred when the amount or concentration of one or a plurality of extracellular RNA species in the plasma, serum or non-cellular bodily fluid is not significantly less than the amount or concentration of one or a plurality of extracellular RNA species found in the reference group.

According to another aspect of the present invention, there is provided methods for detecting, diagnosing, inferring, evaluating or monitoring disease, particularly cancer or neoplastic disease in a human or animal, the method comprising the steps of isolating a predominantly non-cellular fraction of a bodily fluid obtained from a human or animal, wherein the non-cellular fraction may be plasma, serum or other non-cellular bodily fluid from a human or animal; thereafter extracting RNA from the non-cellular fraction of the bodily fluid, such as from plasma, serum or other non-cellular bodily fluid specimen of a human or animal, wherein the extracted RNA may comprise total RNA or a heterogeneous mixture of RNA species or specific RNA species; determining quantitatively or qualitatively the amount or concentration of at least two RNA species from a fraction of said plasma, serum or other non-cellular bodily fluid fraction; wherein comparison of said RNA species thereby detects, diagnoses, infers, or monitors or enables evaluation of a cancer or neoplastic disease or other disease in a human or animal. In a particularly preferred embodiment, at least two of the RNA species are tumor-associated RNA, and cancer or neoplastic disease is detected, diagnosed or inferred or evaluated when the relative or absolute amount or concentration of at least one RNA species from the plasma, serum or non-cellular bodily fluid fraction from a human or animal is greater than the amount or concentration of another RNA species from the plasma, serum, or non-cellular bodily fluid fraction from said human or animal. In another particularly preferred embodiment, at least two of the RNA species are tumor-associated RNA and a third RNA species is not cancer-specific, and cancer or neoplastic disease is detected, diagnosed or inferred or evaluated when the relative or absolute amount or concentration of at least two of the tumor-associated RNA species from the plasma, serum, the tumor-associated RNA species from the plasma, serum, or non-cellular bodily fluid fraction of a human or animal is greater than the amount or concentration of the non-tumor specific RNA species in the plasma, serum, or non-cellular bodily fluid fraction from said human or animal. In another preferred embodiment, at least two of the RNA species are disease-specific, and the disease is diagnosed, detected, inferred, evaluated, characterized, or monitored in a human or animal when the relative or absolute amount or concentration of at least one RNA species from the plasma, serum, or non-cellular bodily fluid fraction is greater than the amount or concentration of another RNA species from the plasma, serum, or non-cellular bodily fluid fraction from said human or animal. In another preferred embodiment, at least two of the RNA species are disease-specific RNA and a third RNA species is not disease-specific, and a disease is detected, diagnosed or inferred or evaluated when the relative or absolute amount or concentration of a least two of the disease-specific RNA species from the plasma, serum, or non-cellular bodily fluid fraction of a human or animal is greater than the amount or concentration of the non-disease specific RNA species in the plasma, serum, or non-cellular bodily fluid fraction from said human or animal.

In another preferred embodiment of the present invention, there is provided methods for detecting, diagnosing, inferring, evaluating or monitoring disease, particularly cancer or neoplastic disease in a human or animal, the methods comprising the steps of isolating a predominantly non-cellular fraction of a bodily fluid obtained from a human or animal, wherein the non-cellular fraction may be plasma, serum, or other non-cellular bodily fluid; thereafter extracting RNA from the non-cellular fraction of the bodily fluid, such as from plasma or serum or other non-cellular bodily fluid specimen of the human or animal (test specimen), wherein the extracted RNA may comprise total RNA or a heterogeneous mixture of RNA species or specific RNA species; determining quantitatively or qualitatively the amount or concentration of one or a plurality of RNA species from a fraction of said test specimen; and determining in a qualitative or quantitative fashion the relative amount or concentration of one or a plurality of extracellular RNA species obtained from the fraction of said test specimen to the amount or concentration of one or a plurality of corresponding RNA species from a reference group or specimen. In one aspect, determination of the quantitative or qualitative amounts or concentrations of RNA species with respect to each other or with respect to a reference group or specimen value or set of values is made on the basis of a statistical analysis. In another aspect said determination with respect to the reference group is provided by either a numerical or positive/negative assessment of each extracellular RNA species within the test specimen relative to the reference group or specimen by setting the reference group or specimen value as the baseline standard. In one aspect of this embodiment, the numerical or positive/negative values assigned to each RNA species from the test specimen using the reference standard to values of other RNA species within the test specimen is made, whereby an RNA expression profile or a qualitative or quantitative set of RNA patterns or a RNA signature is determined, or whereby statistical or similar analysis is performed that thereby detects, diagnoses, infers, evaluates, characterizes, or monitors a cancer or neoplastic disease or other disease of interest in a human or animal. In preferred embodiments, the reference group is a human or human population of individuals without cancer or the disease of interest. In alternative preferred embodiments, the reference group is a human or human population of individuals with cancer or with the disease of interest. In preferred embodiments, the reference group or specimen values or baselines are pre-determined, as previously discussed.

According to another aspect of the present invention, there are provided methods for detecting, diagnosing, inferring, or monitoring cancer or neoplastic disease or other disease in a human or animal, the method comprising the steps of obtaining a plasma or serum or non-cellular bodily fluid fraction from the human or animal; determining in a qualitative or quantitative fashion the amount or concentration of one or more RNA species from a portion of the plasma or serum or non-cellular bodily fluid fraction; evaluating or analyzing in a quantitative or qualitative fashion said amount or concentration of one or more RNA species to that of a reference group, wherein said analysis thereby detects, diagnoses, infers, or monitors a cancer or neoplastic disease or other disease in a human or animal when the quantitative or qualitative amount or concentration of one or more RNA species from said plasma, serum, or non-cellular bodily fluid fraction is either greater or lesser to that demonstrated in a reference group or specimen not having a cancer, neoplastic disease, or other disease of interest, or within the expected range of a reference group with a cancer, neoplastic disease, or other disease of interest.

In preferred embodiments, the reference group is a human or human population of individuals without cancer, and cancer is detected, diagnosed or inferred when the amount or concentration of total extracellular RNA in the fraction of the specimen is greater than the amount or concentration of total extracellular RNA found in the reference group, or when one or more RNA species in the fraction of the specimen is greater than the amount or concentration of said species found in the reference group. In alternative preferred embodiments, the reference group is a human or human population of individuals with cancer, and cancer is detected, diagnosed or inferred when the amount or concentration of total extracellular RNA or one or more RNA species in the fraction of the specimen is not significantly less than the amount or concentration of total extracellular RNA found in the reference group. In an alternative preferred embodiment, the amount or concentration of one or more RNA species may be less than that of the reference group, whereby cancer or neoplastic disease is thereby detected, diagnosed, inferred, evaluated, or characterized. In one aspect of this embodiment, an extracellular messenger RNA amount or concentration within a non-cellular bodily fluid is less than that of a reference group and an extracellular inhibitory or interfering RNA amount or concentration is greater than that of a reference group. In another aspect of this embodiment, an extracellular messenger RNA amount or concentration within a non-cellular bodily fluid is greater than that of a reference group and an extracellular inhibitory or interfering RNA amount or concentration is less than that of a reference group.

In another preferred embodiment, the invention provides methods for detecting, diagnosing, inferring, characterizing, evaluating, or monitoring cancer or neoplastic disease in a human or animal, the method comprising the steps of obtaining a plasma or serum or non-cellular fraction of a bodily fluid from a human or animal; determining directly without prior RNA extraction, the amount or concentration of one or a plurality of extracellular RNA species in a portion of the plasma or serum or non-cellular bodily fluid specimen; quantitatively or qualitatively determining said amount or concentration relative to that of a reference group or specimen, whereby the amount or concentration of RNA species relative to the reference group or specimen detects, diagnoses, infers, or monitors a cancer or neoplastic disease or other disease of interest in a human or animal. In preferred embodiments, the reference group is a human or human population of individuals without cancer, and cancer or neoplastic disease is detected, diagnosed or inferred when the amount or concentration of one or a plurality of extracellular RNA species in the non-cellular bodily fluid of a human or animal is greater than the amount or concentration of one or a plurality of extracellular RNA species found in the reference group or specimen. In alternative preferred embodiments, the reference group is a human or human population of individuals with cancer, and cancer or neoplastic disease is detected, diagnosed or inferred when the amount or concentration of one or a plurality of extracellular RNA species in the non-cellular bodily fluid of a human or animal is not significantly less than the amount or concentration of one or a plurality of extracellular RNA species found in the reference group or specimen.

It is to be explicitly understood that said analysis of two or more RNA species may include quantitative or qualitative analysis of disease-specific RNA to non-disease specific RNA, disease-specific RNA to disease-specific RNA, non-disease specific RNA to non-disease specific RNA, non-mutated tumor RNA to mutated or non-mutated tumor RNA; tumor RNA to non-tumor RNA; coding RNA to coding RNA; coding RNA to non-coding RNA; and non-coding RNA to non-coding RNA; or any combination thereof. There is further a need in the art for methods of enhancing the reliability and reproducibility of said analysis by maintaining the relative or absolute amounts, concentrations, or ratios of the various RNA species over time.

In a preferred embodiment of the inventive methods, the bodily fluid is blood, plasma, serum, urine, effusions including pleural effusions, ascitic fluid, saliva, cerebrospinal fluid, gastrointestinal secretions, bronchial secretions including sputum, cervical secretions, or breast secretions. In a particularly preferred embodiment, the bodily fluid is plasma or serum. In particularly preferred embodiments of the inventive methods, the non-cellular (acellular) bodily fluid or bodily fluid fraction is blood plasma or serum. Other predominantly non-cellular (acellular) bodily fluids particularly include urine, saliva, and cerebrospinal fluid, and may further include certain gastrointestinal secretions and transudates.

In preferred embodiments a predominantly non-cellular fraction of a bodily fluid is isolated by obtaining a bodily fluid from a human or animal and centrifuging the bodily fluid to isolate a predominantly non-cellular fraction of a bodily fluid. In another preferred embodiment, a predominantly non-cellular fraction of a bodily fluid is isolated by obtaining a bodily fluid from a human or animal and passing the bodily fluid through a filter of sufficient size to separate the cellular and non-cellular fractions of the bodily fluid, or otherwise size-fractionate the bodily fluid, and thereby isolate the non-cellular fraction of a bodily fluid. In particularly preferred aspects of these embodiments, plasma or serum is isolated from blood. Further, predominantly non-cellular bodily fluids, including but not limited to urine, saliva, and cerebrospinal fluid may be further purified by process of centrifugation, or filtering or size-fractionation, or analyzed without further purification.

In preferred embodiments of the inventive methods, the amount of total extracellular RNA, or one or a plurality of extracellular RNA species, is determined quantitatively or qualitatively using a method that is nucleic acid amplification, signal amplification, spectroscopy including mass spectroscopy, or hybridization, preferably to a detectably-labeled probe.

In preferred embodiments of the inventive methods, RNA is extracted from blood, plasma, serum, or other bodily fluid or from the non-cellular fraction of a bodily fluid using an extraction method known to the art that includes but is not limited to a gelatin extraction method; a silica, glass bead, or diatom extraction method; guanidinium thiocyanate acid-phenol based extraction methods; guanidinium thiocyanate acid based extraction methods; phenol-chloroform based extraction methods; by centrifugation through a cesium chloride or similar gradient; or using commercially-available RNA extraction methods, most preferably as provided in a kit comprising instructions from the kit manufacturer.

In preferred embodiments of the invention, RNA extracted from plasma, serum, or other bodily fluid is reverse transcribed to cDNA prior to hybridization and detection or hybridization, amplification and detection. In these embodiments, the amount or concentration of RNA is determined by qualitative or quantitative analysis of cDNA or amplified cDNA product or amplified signal.

In preferred embodiments of the invention, extracted RNA or the corresponding cDNA is amplified qualitatively or quantitatively to determine the amount or concentration of a RNA species, using an amplification method that is, for example, polymerase chain reaction, or reverse transcriptase polymerase chain reaction; ligase chain reaction; DNA or RNA signal amplification; amplifiable RNA reporter methods; Q-beta replication; transcription-based amplification; isothermal nucleic acid sequence based amplification; self-sustained sequence replication assays; boomerang DNA amplification; strand displacement activation; cycling probe technology; and any combination or variation thereof.

In preferred embodiments of the inventive methods, detection of amplified RNA or cDNA product is performed using a detection method that is, for example, gel electrophoresis; enzyme-linked immunosorbent assay (ELISA), including embodiments comprising biotinylated or otherwise modified amplification primers as known to the art; hybridization using a specific, detectably-labeled probe, for example but not limitation, a fluorescent-, radioisotope-, or chromogenically-labeled probe; Southern blot analysis; Northern blot analysis; electrochemiluminescence; reverse dot blot detection; high-performance liquid chromatography; and variations thereof.

The methods of the invention particularly provide methods for identifying humans at risk for developing a disease, particularly cancer or other neoplastic disease, or who have a malignancy or premalignancy. The methods of the invention thus provide methods for identifying humans having a malignancy such as breast, ovarian, lung, cervical, colorectal, gastric, pancreatic, bladder, endometrial, brain, kidney, or esophageal cancers, leukemias, lymphomas, melanoma, or sarcomas; and premalignancies including but not limited to colorectal adenoma, cervical dysplasia, cervical intraepithelial neoplasia (CIN), bronchial dysplasia, atypical hyperplasia of the breast, ductal carcinoma in-situ, atypical endometrial hyperplasia, and Barrett's esophagus.

The invention thus permits the presence of cancerous (malignant) or pre-cancerous (premalignant) cells within a human or animal to be detected or inferred by determining an amount or concentration of RNA in the plasma, serum, or other bodily fluid of said human or animal that exceeds the amount or concentration normally present in the plasma, serum, or other bodily fluid of a human or animal without cancer or pre-malignancy.

The invention also permits the existence of a disease within a human or animal to be detected or inferred by determining an amount or concentration of RNA in the plasma, serum, or other bodily fluid of said human or animal that exceeds the amount or concentration normally present in the plasma, serum, or other bodily fluid of a healthy human or animal.

An advantageous application of this invention is to identify humans or animals with disease.

It is a particularly advantageous application of this invention to identify humans or animals having cancer.

Another advantageous application of this invention is to identify humans or animals having risk for developing cancer.

Another advantageous application of this invention is to identify humans or animals having a premalignant disease.

Another advantageous application of this invention is for monitoring cancer, including response to cancer therapies, including surgery, biotherapy, hormonal therapy, anti-sense therapy, monoclonal antibody therapy, chemotherapy, vaccines, anti-angiogenic therapy, cryotherapy, radiation therapy, and therapies based upon or directed at inhibitory or interfering RNA or regulatory RNA, including siRNA and miRNA.

Another advantageous application of this invention is selecting humans or animals for cancer therapies, including surgery, biotherapy, hormonal therapy, anti-sense therapy, monoclonal antibody therapy, chemotherapy, vaccines, anti-angiogenic therapy, cryotherapy, radiation therapy, and therapies based upon or directed at inhibitory or interfering RNA or regulatory RNA, including siRNA and miRNA.

Another advantageous application of this invention is to provide a marker as a guide to whether adequate therapeutic effect has been achieved, or whether additional or more advanced therapy is required, and to assess prognosis in a patient.

Another advantageous application of this invention is to provide an indicator of a relapsed cancer following therapy, or impending relapse, or treatment failure.

Another advantageous application of this invention is to identify humans or animals who might benefit from additional diagnostic procedures, wherein said procedures include but are not limited to surgery, biopsy, needle aspiration, radiologic imaging including X-ray, MRI, and CT scanning, radionucleotide imaging, colonoscopy, sigmoidoscopy, bronchoscopy, endoscopy, PET scanning, stool analysis, sputum analysis, cystoscopy, pelvic examination, and physical examination.

The invention further provides kits that provide stabilizing agent for use in combination with plasma, serum, or bodily fluid to stabilize extracellular RNA within said plasma, serum, or bodily fluid.

The invention also provides kits enabling quantitative or qualitative assessment of total RNA or specific RNA species in plasma or serum or non-cellular bodily fluid fractions, wherein components for bodily fluid collection, RNA extraction, amplification or signal amplification, and RNA detection may be provided, and further wherein a reference range for normal values or cancer values or a reference specimen may be provided to enable identification or selection of a human or animal with or at risk for cancer.

The present invention thus provides methods to enhance the intra-specimen stability and reproducibility of the ratio between two or more RNA species in a plasma or serum or bodily fluid specimen, and kits thereof.

Specific preferred embodiments of the present invention will become evident from the following more detailed description of certain preferred embodiments and the claims.

### DETAILED DESCRIPTION OF THE INVENTION

This invention relates to methods for detecting, diagnosing, inferring, evaluating, or monitoring cancer or neoplastic disease or other disease of interest in a human or animal by stabilizing the extracellular RNA in plasma, serum, or non-cellular bodily fluid, and thereafter assessing the amount or concentration of RNA in said plasma, serum, or other non-cellular bodily fluid of the human or animal, and determining in a quantitative or qualitative fashion the amount or concentration of one or more RNA species in said plasma, serum, or non-cellular bodily fluid isolated from the bodily fluid of a human or animal relative to the amount or concentration of one or more other RNA species in said bodily fluid, or relative to the amount or concentration of RNA found in bodily fluid from a reference individual, group or population or specimen of known disease status. In particular, the invention provides methods for diagnosing, detecting, inferring, evaluating, characterizing, or monitoring the presence of cancerous or precancerous cells in a human or animal, whether from a non-hematologic neoplasm (i.e., a solid-tumor) or from a hematologic malignancy (such as leukemia, lymphoma, myeloma, etc.).

The methods of the invention in the first step isolate a predominantly non-cellular fraction of a bodily fluid obtained from a human or animal, wherein the non-cellular bodily fluid fraction may be plasma, serum, or other bodily fluid. In preferred embodiments, the non-cellular fraction is isolated by centrifugation of a cellular bodily fluid such as blood, or by other means known to the art such as but not limited to by passing the bodily fluid through a filter or otherwise size fractionating or density fractionation to separate the bodily fluid, whereby the cellular and non-cellular components of the bodily fluid are separated and the non-cellular fraction of the bodily fluid is thereby isolated. In particular preferred embodiments, plasma or serum is isolated from whole blood. Serum is obtained by allowing blood to clot, and may thereafter by isolated or further purified by methods known in the art or described herein. It is recognized that the non-cellular fraction of a bodily fluid may pass through multiple steps to further purify or isolate a non-cellular fraction, for example by differential centrifugation, or by combining centrifugation with filtering or size exclusion, or weight/density separation. Furthermore, it will be recognized that some bodily fluid such as transudates, saliva, urine, and cerebrospinal fluid may be predominantly non-cellular, whereby a predominantly non-cellular fraction of bodily fluid may be obtained by obtaining the bodily fluid from a human or animal. It is further to be recognized that predominantly non-cellular bodily fluids may be further purified by centrifugation, filtering, or size-fractionation of the non-cellular fluid. It is to be recognized herein that plasma and serum are considered bodily fluids, and further are considered to be non-cellular fractions of a bodily fluid being blood. In preferred embodiments, the bodily fluid is blood, plasma, serum, urine, effusions including pleural effusions, ascitic fluid, saliva, cerebrospinal fluid, gastrointestinal secretions, bronchial secretions including sputum, cervical secretions, or breast secretions. Plasma and serum are particularly preferred bodily fluids, but any bodily fluid a portion of which comprises extracellular RNA, and particularly tumor-associated extracellular RNA is useful in the practice of the methods of this invention.

In a preferred embodiment, the methods of the invention in the second step stabilize the extracellular RNA in plasma, serum or non-cellular bodily fluid by combining the plasma, serum, or non-cellular bodily fluid with an agent that protects or stabilizes RNA from degradation by RNase, herein referred to as stabilizing or stabilizer agent. The invention allows for this second step to be an optional step of the invention, depending upon the need or desire to stabilize the RNA. Thereafter, in the remaining steps of the invention, there is determined an amount, concentration or other quantitative or comparative assessment of RNA from plasma, serum, or other non-cellular bodily fluid specimen obtained from a human or animal, wherein the RNA can be either total extracellular RNA, or one or a plurality of specific RNA species or multiple specific RNA species. In the method of the invention, quantitative or qualitative determination of RNA amount or concentration in plasma, serum, or non-cellular bodily fluid generally comprises the step of extracting RNA from the non-cellular fraction of the bodily fluid, wherein the extracted RNA may comprise total RNA or a heterogeneous mixture of RNA species or specific RNA species; followed by quantitative or qualitative amplification or signal amplification of one or more RNA species extracted from the non-cellular bodily fluid, generally using primers or probes to the RNA species of interest or to cDNA reverse transcribed therefrom; followed by quantitative or qualitative detection of the amplified or signal amplified product. In the last step of the methods of the invention, a quantitative or qualitative assessment is made of the amount or concentration or ratio of one or more RNA species is made to other RNA species, or total RNA, or extracellular DNA, or extracellular protein within the plasma, serum, or non-cellular bodily fluid specimen from the human or animal, or to a reference group, specimen, or value, to thereby determine, diagnose, detect, evaluate, characterize, or monitor cancer or a neoplastic disease or another disease of interest.

As used herein, the term “RNA species” refers to RNA selected from one or more of the groups comprising messenger RNA (mRNA), transfer RNA (tRNA), ribosomal RNA (rRNA), mitochondrial RNA (mtRNA), inhibitory or interfering RNA such as short interfering RNA (siRNA) or micro RNA (miRNA), coding RNA, non-coding RNA, RNA having a sequence complimentary to a mutated or altered DNA, RNA having a sequence complimentary to non-mutated DNA, ribonucleoprotein RNA, and messenger RNA splice variants. It is understood herein that RNA species may further be disease-associated, tumor-associated, disease-related, tumor-related, and non-tumor-related. RNA species of interest will be dependent upon the disease of interest, and when evaluating for cancer, may be either tumor-related RNA or non-tumor related RNA. Total extracellular RNA will be recognized as comprising both tumor-related and non-tumor-related RNA when obtained from a patient with cancer or other neoplastic disease.

As noted, this invention further provides methods for detecting, diagnosing, inferring, evaluating, characterizing, or monitoring cancer or neoplastic disease or other disease of interest in a human or animal without requiring a stabilizing or stabilizer agent. The method of the invention in the first step isolates a predominantly non-cellular fraction of a bodily fluid obtained from a human or animal, wherein the non-cellular bodily fluid may be plasma, serum, or other non-cellular bodily fluid. Isolation of the non-cellular bodily fluid is performed as previously described, such as by centrifugation or size fractionation of a bodily fluid. Thereafter, RNA is extracted from the non-cellular fraction of the bodily fluid, wherein the extracted RNA may be performed by extracting total RNA from plasma, serum, or non-cellular bodily fluid of the human or animal, or by extracting various heterogeneous RNA species or specific RNA species in a more specific manner, for example but not limitation by using hybridizing probes or coated beads including magnetic beads, or coated solid substrates upon which RNA or cDNA species may be hybridized. Extracellular RNA may further be extracted from the non-cellular bodily fluid by first extracting or isolating the particles and apoptotic bodies containing said RNA from the non-cellular bodily fluid, such as by employing centrifuging at high speeds, such as but not limited to greater than 50,000×g and particularly greater than 100,000×g, or by extracting apoptotic bodies with proteins or antibodies that bind to the apoptotic body, wherein said proteins or antibodies may further be attached to beads or magnetic spheres or solid substrates. Particles and apoptotic bodies containing RNA may further be isolated for the non-cellular bodily fluid by passing the non-cellular bodily fluid through various appropriately sized filters and by similar methods of size fractionation, and by chemical or mechanical gradients or columns. Following isolation of the particles and apoptotic bodies, the RNA may be directly extracted from the particles or apoptotic bodies by the methods as described herein for extracting RNA from non-cellular bodily fluid. The inventive steps thereafter assess the amount or concentration of RNA species, particularly extracellular RNA species, in said plasma, serum, or other non-cellular bodily fluid of the human or animal, wherein in the preferred step one or more RNA species extracted from the non-cellular bodily fluid are amplified or signal amplified or hybridized in a quantitative or qualitative manner, preferably using primers or probes specific for the RNA species of interest, or cDNA derived therefrom, followed by quantitative or qualitative detection of the amplified or signal amplified or hybridized product. The amount or concentration of one or more RNA species in said plasma, serum, or non-cellular bodily fluid obtained from the human or animal is thereby determined, and a quantitative assessment is made relative to the amount, concentration, or ratio of one or more RNA species to another RNA species, or to total RNA, or extracellular DNA, or extracellular protein, within the plasma, serum, or non-cellular bodily fluid specimen from the human or animal, or to a reference group, specimen, or value to thereby determine, diagnosis, evaluate, or monitor cancer, neoplastic disease, or another disease of interest. Other diseases of interest include but are not limited to cardiovascular, neurological, endocrine, pulmonary, gastrointestinal, and obstetrical/gynecological diseases and conditions.

Qualitative or quantitative determination of the amount or concentration of one or more RNA species from said human or animal's non-cellular bodily fluid specimen allows an absolute or relative assessment to one or more other RNA species from said non-cellular bodily fluid, or to total RNA, extracellular DNA, or extracellular protein from said non-cellular bodily fluid, or to a standard amount, concentration, or value or set of values from a reference individual, group, population, or specimen. Said assessment relative to a reference standard can be made on the basis of a previously-determined reference set of values for said individual, group or population or specimen, or alternatively upon a newly determined reference set of values or standards for the individual, group, or population or specimen. The reference set of values or standards may include standard deviations, confidence intervals, ranges based upon age, sex, race, disease stage or condition, disease characteristic, prior treatment characteristics, or other parameters. Assessment of the relative or absolute concentration or amount of extracellular RNA species in a non-cellular bodily fluid from a human or animal to the amount or concentration of said RNA species in said bodily fluid from a reference individual, group, or population or specimen or other standard thereby enables determination of the likelihood that the subject human or animal has a disease. In one aspect a disease such as cancer or neoplastic disease such as premalignancy is determined or evaluated, wherein if the amount or concentration of one or a plurality of specific RNA species from the non-cellular bodily fluid fraction of the subject human or animal is demonstrated to be either greater than or less than the amount or concentration present in individuals, groups, or populations without disease, then a disease (for example, cancer) or an increased risk of disease (for example, due to the existence of a premalignancy) will be inferred in the human or animal subject. Similarly, if the amount or concentration of total extracellular RNA, or of one or a plurality of specific RNA species thereof in the bodily fluid of the subject is within the range of a group or population with a disease, in one aspect cancer or neoplastic disease such as a premalignancy, then a disease, (for example, cancer) or an increased risk of disease (for example, due to the existence of a premalignancy) will be inferred in the human or animal subject. If the amount or concentration of total extracellular RNA, or of one or a plurality of specific RNA species thereof in the non-cellular bodily fluid of the subject is less than the range for patients with a disease (for example, cancer), or within the range of the healthy population, then the risk of disease, in one aspect cancer or an increased risk of disease (for example, due to the existence of a premalignancy) will be less. It will be recognized that the limits of the reference range values may be set in a manner that determines a sensitivity or specificity or positive predictive value or negative predictive value for the assay, or otherwise provides the probability of the assay correctly identifying a subject with a disease such but not limited to cancer or neoplasm. Thus, in this manner the reference range for a group or population can be defined that increases the sensitivity or specificity of the assay. It will also be recognized that lower concentrations of some extracellular RNA species relative to the reference group may be indicative of higher risk of malignancy. Determination of the quantitative or qualitative amounts or concentrations of extracellular RNA species with respect to each other or with respect to a reference group or specimen value or set of values can be made on a visual, numerical, or mathematical assessment, for example but not limitation on the basis of a statistical analysis.

The invention provides for the determination of values, standards, or baselines for the amounts or concentrations of particular extracellular RNA species, or total extracellular RNA, or extracellular RNA patterns, extracellular RNA expression profiles, or extracellular RNA signatures of a reference group, reference population, or reference specimen by the evaluation of extracellular RNA in non-cellular bodily fluids obtained from the defined reference group, population, or specimen. The extracellular RNA would be quantitatively or qualitatively evaluated for each subject of the reference group, population, or specimen group in the same manner described herein for analysis of extracellular RNA in the bodily fluid of “test” human or animal subjects. In the preferred embodiment, the quantitative or qualitative results are then statistically evaluated to provide the values, range including minimum range, maximum range, mean and median, standard, deviations, and/or confidence intervals for the whole reference group, population, or specimen group.

It is to be recognized that a variety of individuals, groups, or populations will provide suitable reference values that enable discrimination of abnormal (disease-, and more particularly cancer-, related) and normal amounts or concentrations of total extracellular RNA, or of one or a plurality of specific RNA species thereof in the bodily fluid of the subject. Appropriate reference individuals, groups, or populations include but is not limited to: a healthy human or animal, more specifically a human or animal population without cancer or without neoplastic disease (cancer and/or premalignancy) or other disease of interest; a human or animal population with a disease of interest, in one aspect more specifically a human or animal population with neoplastic disease (cancer and/or premalignancy) or a human or animal population with cancer; a previously-isolated bodily fluid specimen from the human or animal under evaluation corresponding to a known disease or health state. In addition, it will be recognized that certain defined groups or populations will provide useful reference values to assess probability of disease, particularly cancer or premalignancy, in a subject, including but not limited to: groups and populations defined by age or gender and the presence or absence of disease, particularly cancer or premalignancy; groups and populations defined by race or ethnicity and presence or absence of disease, particularly cancer or premalignancy; groups and populations defined by non-neoplastic diseases; groups and populations defined by specific tumor types; groups and populations defined by stage or extent of cancer of a particular type; groups and populations defined by certain life-style risks or environmental or occupational risks for cancer, such as smokers or workers occupationally exposed to carcinogens; and groups and populations defined by genetic or family risk for cancer. It is to be understood that the assessment of the subject's extracellular RNA species of interest, or the subject's total extracellular RNA in a bodily fluid such as blood plasma or serum to reference groups and populations may be made by either non-statistical or statistical analysis, as is known to the art.

It is further to be understood that quantitative or qualitative analysis of multiple extracellular RNA species from a non-cellular bodily fluid of a human or animal enables determination of a RNA pattern or RNA expression profile or RNA signature, wherein said pattern, profile, or signature thereby enables the diagnosis, detection, evaluation, characterization, or monitoring of a cancer, neoplastic disease, or other disease of interest. The resultant RNA pattern, RNA expression profile, or RNA signature may be analyzed visually, statistically, or otherwise mathematically against the known RNA pattern, RNA expression profile, or RNA signature of a reference group or specimen having or not having the cancer, neoplastic disease, or disease of interest. The reference group or specimen RNA pattern, RNA expression profile, or RNA signature may be specific to a particular cancer, or be intended to encompass various cancer types.

In particularly preferred embodiments of the invention, one or more RNA species present in plasma, serum, or non-cellular fraction of a bodily fluid of a human or animal is quantitatively or qualitatively assessed relative to one or more other RNA species within said plasma, serum, or non-cellular fraction of a bodily fluid, whereby assessment of said RNA species is made by either non-statistical or statistical analysis, as known in the art. Quantitative or qualitative analysis of two or more extracellular RNA species in plasma, serum, or other bodily fluid thereby provides methods for detecting, inferring, characterizing, evaluating, or monitoring cancer or premalignancy or another disease of interest. In one preferred aspect of this embodiment, an invention step stabilizes the extracellular RNA in plasma, serum, or non-cellular bodily fluid by combining RNA plasma, serum, or bodily fluid with an agent that protects or stabilizes RNA from degradation by RNase, herein referred to as stabilizing agent. In another preferred embodiment, the stabilizing agent is not utilized prior to RNA extraction from a non-cellular bodily fluid. Within both embodiments, thereafter the step or steps that follow determine an amount, concentration, or other quantitative or comparative assessment of the extracellular RNA species of interest, in the method previously described wherein. Assessment may be accomplished by extracting RNA from the plasma, serum, or non-cellular bodily fluid; amplifying or signal amplifying either sequentially or concurrently and in a qualitative or quantitative fashion the RNA species of interest, or cDNA derived therefrom, comprising a fraction of the extracted RNA; detecting the amplified products or amplified signal of the RNA species or cDNA derived therefrom; whereby detection, diagnosis, evaluation, characterization, or monitoring of cancer or premalignancy is thereby accomplished. Analysis of two or more RNA species from a plasma, serum, or other bodily fluid specimen, particularly non-cellular bodily fluid may be further performed by comparing the absolute amount or relative amount or concentration or the ratio of at least one RNA species to another RNA species of interest within said bodily fluid specimen, or to one or more reference RNA species or values from a reference group or specimen. Analysis may further be performed by quantitatively or qualitatively assessing one or more RNA species from a plasma, serum, or other bodily fluid specimen, particularly non-cellular bodily fluid, to total RNA or extracellular DNA or extracellular protein from the specimen, or to total RNA or extracellular DNA or extracellular protein from a reference group or specimen. The methods of the invention allow quantitative or qualitative analysis of an extracellular RNA species in plasma, serum, or other bodily fluid of a human or animal to the corresponding protein to said RNA species within said specimen.

In particularly preferred embodiments of the invention, the non-cellular bodily fluid fraction is blood plasma or serum. Either fresh (i.e., never frozen) blood plasma or serum, or frozen (stored) and subsequently thawed plasma or serum may be used for purposes of these embodiments. In those preferred embodiments when stabilizing agent is not used, the non-cellular component of blood (i.e., plasma or serum) is isolated soon after drawing, preferably within 24 hours and most preferably within 6 hours, to minimize any degradation of extracellular nucleic acids or release of RNA from blood cells. While early isolation of the non-cellular bodily fluid fraction is not a requirement of the methods of the invention, it will be recognized that variations of early isolation can be employed as set forth below, without limitation implied. In one aspect, the whole blood may be initially processed to stabilize the RNA or to stabilize phospholipids encapsulating the extracellular RNA, or to inhibit nucleases present in blood. In a preferred embodiment, plasma or serum obtained from blood may be processed to stabilize the RNA or to stabilize phospholipids encapsulating the extracellular RNA, or to inhibit nucleases present in the specimen. Stabilizing agents including nuclease inhibitors when added after separation of plasma or serum enable nuclease to degrade any free RNA released from cells during the centrifugation of whole blood. Stabilizing agents or inhibitors may be provided within kits according to the invention or within venipuncture collection tubes or devices. Such initial processing is useful if specimen transport or work schedules will result in processing delays. In another aspect of the practice of the invention, initial processing of either whole blood or plasma or serum may be performed by hybridizing associated apoptotic bodies or other RNA encapsulated particles in the whole blood, plasma, or serum, to solid substrates shortly after venipuncture, preferably using reagents provided in a kit of this invention or as part of specialized blood collection systems, or after centrifugation or other separation of plasma or serum from whole blood. It is preferred that the processing of the specimen from the human or animal subject and from the reference group or population be handled in a similar or like manner to the extent practical, or alternatively the effect due to variations in specimen processing be defined and reference values be appropriately adjusted, when analysis will be based upon quantitative reference values or specimens.

In a preferred embodiment, blood is first collected by venipuncture and may be kept on ice or at room temperature until serum or plasma is separated from whole blood as previously described, for example using centrifugation methods. While a considerable range of centrifugation speeds may be employed, centrifugation at high speeds (such as beyond 50,000×g and particularly beyond 100,000×g) for prolonged periods should be avoided to prevent clearance of RNA-containing apoptotic bodies or other encapsulated extracellular RNA particles from the supernatant. Non-limiting examples of suitable conditions is centrifuging a blood specimen at a range of from 300 to 5,000×g for five to thirty minutes, and most preferably between 400 and 3000×g for five to twenty minutes, however centrifugation at other speeds and durations and fractionating by other standard methods to produce plasma or serum will suffice. High speeds of centrifugation may be employed if it is desired to extract extracellular RNA directly from the pellet, rather than from the supernatant. Sera or plasma obtained in the above manner can be assayed directly or stored either at room temperature or stored refrigerated or stored frozen at below 0 degrees centigrade, for example at −20 to −80 degrees centigrade, until further analysis according to the methods of this invention.

In a preferred embodiment, RNA in plasma, serum, or non-cellular bodily fluid is stabilized against RNase by mixing the plasma, serum, or bodily fluid with an RNase inhibitor agent or RNase inactivator. The plasma or serum or blood may be mixed with the stabilizing agent shortly following venipuncture. Alternatively, the plasma or serum may be mixed with the stabilizing agent following its isolation, such as by filtration, centrifugation or clotting. It is most preferred but not required that plasma or serum be mixed with the stabilizing agent within 24 hours of blood draw and most preferably within 6 hours of blood draw. In a preferred embodiment, the plasma or serum RNA so stabilized is total RNA, a portion of which comprises non-mutated RNA. In one aspect a portion of said RNA further comprises a coding RNA and/or a non-coding RNA. In another aspect a portion of said RNA comprises one or more RNA species within the group comprising mRNA, rRNA, tRNA, mtRNA, siRNA, miRNA and ribonuclease RNA. RNase inhibitor or inactivating agents are known to the art, including commercially available agents, and may be utilized as stabilizing agent. In a particularly preferred embodiment, plasma or serum is mixed 1:1 with Trizol (Life Technologies, Carlsbad, Calif.) as a stabilizing agent.

The stabilizing agent may be provided within a kit to be used for the stabilization of extracellular RNA. The components of the kit would include a stabilizing agent such as Trizol or similar RNase inhibitor, usually of a predetermined amount, and may further include one or more additional kit components including but not limited by a tube or vacutainer collection tube for venipuncture, wherein said vacutainer tube may or may not contain the stabilizing agent, and components including RNA extraction agent, primers or probes to specific RNA or cDNA species, reagents for amplification or signal amplification or reverse transcription; and/or a RNA reference specimen. The kit may further include components that are solid surfaces upon which qualitative or quantitative detection of RNA or cDNA or their amplified or signal amplified products may be performed, or may include reagents such as but not limited to antibodies for detecting corresponding proteins. It is to be recognize that a kit that provides reagents to enable practice of the invention may be provided that comprises the one or more of the above kit components in the absence of a stabilizing agent.

The invention further encompasses other stabilizing agents that stabilize or protect plasma, serum, or bodily fluid extracellular RNA from RNase. In a preferred embodiment, the stabilizer agent hybridizes to specific RNA species, thereby stabilizing the RNA against nucleases. In another preferred embodiment, the stabilizer agent is a protein that binds to the RNA species in a manner that stabilizes the RNA species against degradation by RNase. In another preferred embodiment of the invention, the stabilizer agent increases the stability of a phospholipid membrane, thereby stabilizing the particle or apoptotic body encapsulating extracellular RNA in plasma, serum, or bodily fluid. In another embodiment, the stabilizer agent promotes aggregation of particles or apoptotic bodies encapsulating extracellular RNA in plasma or serum or bodily fluid. The stabilizing agent may be a coated bead or particle, or a coated solid surface. Similarly, kits comprising the stabilizer agent, as described above, which may further include one or more additional kit components including but not limited to a vacutainer tube for venipuncture, wherein said vacutainer tube may or may not contain the stabilizing agent, RNA extraction agent, primers or probes to specific RNA or cDNA species, reagents for amplification or reverse transcription, and/or an RNA reference specimen. The kit may further include components that are solid surfaces upon which qualitative or quantitative detection of RNA or cDNA or their amplified or signal amplified products may be performed, or may include reagents such as but not limited to antibodies for detecting corresponding proteins.

In a preferred embodiment of the invention, extracellular RNA in plasma or serum or other bodily fluid of the human or animal is assayed by extracting total extracellular RNA from plasma or serum or other non-cellular bodily fluid fraction of the human or animal, determining quantitatively or qualitatively the amount or concentration of total RNA, or one or a plurality of specific RNA species thereof comprising a portion of the total RNA, and determining quantitatively or qualitatively the amount or concentration obtained from the human or animal relative to the total extracellular RNA, or one or a plurality of specific RNA species thereof from a reference group, wherein said determination detects, diagnoses, infers, or monitors a disease, particularly cancer or neoplastic disease in the human or animal. Bodily fluids are preferably separated into essentially cellular and non-cellular components, using centrifugation or other fractionation techniques, and total extracellular RNA thereafter extracted from the non-cellular components.

In the practice of the methods of this invention, total RNA can be extracted from bodily fluid using methods well-known to the art, including but not limited to gelatin extraction method; silica, glass bead, or diatom extraction method; guanidinium thiocyanate acid-phenol based extraction methods; guanidinium thiocyanate acid based extraction methods; centrifugation through a cesium chloride or similar gradient; phenol-chloroform based extraction methods; hybridization and immunobead separation; or commercially available RNA extraction methods. Methods of RNA extraction are further provided in U.S. Pat. No. 6,329,179 B1, incorporated herein in its entirety by reference. If plasma or serum had been previously frozen, upon assay in the preferred embodiment it is thawed rapidly, for example in a warm water bath at about 37 degrees centigrade, and thereafter RNA rapidly extracted to minimize degradation thereof.

However, it should be understood that extraction of total RNA from the non-cellular bodily fluid fraction is not a requirement for the practice of the methods of this invention. In some embodiments, methods such as spectroscopic methods including mass spectroscopy, and cytometry can be used for direct analysis of total extracellular RNA or RNA encapsulated particles within the bodily fluid, or specific RNA species may be extracted separately from plasma or serum or other bodily fluid by hybridizing to other agents, including but not limited to other oligonucleotides attached to beads, or magnetic particles or solid surface substrates, and thereby extracted.

The amount or concentration of RNA from the non-cellular bodily fluid is determined quantitatively or qualitatively using nucleic acid (RNA or cDNA) amplification, signal amplification, spectroscopy including mass spectroscopy, or hybridization to a detectably-labeled probe. In a preferred embodiment, a portion of the RNA extracted from plasma, serum, or a non-cellular bodily fluid fraction is amplified or signal amplified qualitatively or quantitatively. RNA extracted from blood plasma or serum or other bodily fluid may first be reverse transcribed to cDNA, whereupon the cDNA is amplified or signal amplified qualitatively or quantitatively. In preferred embodiments, amplification is performed using primers or probes that are specific for particular RNA or cDNA species, wherein the RNA or its cDNA may be a non-tumor related RNA or a tumor-related RNA. Non-tumor RNA include but are not limited to housekeeper gene RNA, and non-limiting examples of non-tumor RNA include RNA encoding all or a portion of c-abl, porpho-bilinogen deaminase (PBDG), glyceraldehyde 3-phosphate dehydrogenase (GAPDH), retinoic acid receptor (RAR), and beta-actin. Examples of tumor-related or tumor-associated RNA not intending to be limiting include tyrosinase RNA, keratin RNA species, prostate specific antigen RNA, alpha-fetoprotein RNA, BCR/abl RNA, carcinoembryonic antigen RNA, p97 RNA, p16 RNA, MUC 18 RNA, PML/RAR RNA, CD44 RNA, EWS/FLI-1 RNA, EWS/ERG RNA, AML1/ETO RNA, MAGE RNA species, beta human chorionic gonadotropin RNA, telomerase-associated RNA including TEP 1 RNA, human telomerase RNA template (hTR) RNA and telomerase reverse transcriptase protein (hTERT) RNA, bcl-2 RNA, bax RNA, survivin RNA, COX-2 RNA, P53 RNA, c-myc RNA, her-2/neu RNA, Von Hippel-Lindau gene RNA, retinoblastoma gene RNA, mutated in colon cancer (MCC) gene RNA, adenomatous polyposis coli (APC) gene RNA, deleted in colon cancer (DCC) gene RNA, epidermal growth factor receptor (EGFR)RNA, epidermal growth factor (EGF) RNA, hn RNP-A1 RNA, hn RNP-A2/B1 RNA, hn RNP-K RNA, 5T4 RNA, DNA methyltransferase RNA, matrix metalloproteinase species RNA, mammaglobin RNA, DD3(PCA3) RNA, glutathione S-transferase RNA, MDR-1 RNA, and JC virus RNA. It will be recognized that the above examples are not intended to be limiting, and any non-tumor or tumor-related RNA species or corresponding cDNA may be detected according to the methods of this invention for the detection, evaluation, or characterization of cancer. Further, other RNA species or their cDNA will be employed corresponding to the particular disease of interest. Further, it will be recognized that various RNA species are well known to the art, and that the scope of the invention is meant to encompass these RNA species without limitation.

Various amplification methods or signal amplification methods are known in the art and can be used in accordance with the methods of this invention. In preferred embodiments of the methods of the invention, quantitative or qualitative amplification is performed using an amplification or signal amplification method such as but not limited to polymerase chain reaction; reverse transcriptase polymerase chain reaction; ligase chain reaction; DNA or RNA signal amplification; branched-chain amplification; amplifiable RNA reporters; Q-beta replication; transcription-based amplification; isothermal nucleic acid sequence based amplification; self-sustained sequence replication assays; boomerang DNA amplification; strand displacement activation; cycling probe technology; or any combination or variation thereof. In one aspect of this embodiment, quantitative amplification is performed using the Taqman technology (Perkin Elmer Biosystems) and real-time qPCR using the ABI PRISM 7700 sequence detection system (Applied Biosystems), with primers for the target RNA using a dye-labeled internal primer, or other fluorescent PCR-based assays. In another aspect of this embodiment, quantitative amplification is performed using competitive PCR with the MassARRAY process (Sequenom, Inc., San Diego, Calif.), and other MALDI-TOF processes. Any of the quantitative amplification approaches known in the art are similarly applicable to the methods of the invention.

In preferred embodiments, following amplification the RNA or cDNA amplified or signal amplified product is detected in a quantitative or qualitative manner by methods known to the art. In preferred embodiments of the inventive methods, detection of amplified RNA or cDNA product is performed using a detection method selected from a group consisting of gel electrophoresis; ELISA detection including modifications, including biotinylated or otherwise modified primers; hybridization using a specific, fluorescent-, radioisotope-, or chromogenically-labeled probe; Southern blot analysis; Northern blot analysis; electrochemiluminescence; reverse dot blot detection; and liquid chromatography, including high-performance liquid chromatography.

Upon amplification and detection of total extracellular RNA or one or a plurality of specific RNA species, most preferably wherein one or a plurality of species of total extracellular RNA is a disease- or tumor-related gene, an amount or concentration or other value permitting a quantitative or qualitative assessment of said extracellular RNA, or cDNA therefrom, is determined, using for example but not intending to be limiting, gel intensity, signal intensity, or color intensity, color, mass, or electrical propensity. Assessment is further made relative to either other RNA species, or DNA species, or protein species, including total RNA or total DNA, within said specimen. Alternatively, qualitative or quantitative assessment is made of the extracellular RNA species to a reference individual, group, or reference population or reference specimen based upon analysis of said RNA under similar condition and methods, or by extrapolation to similar conditions and methods.

In another embodiment of the invention, determination of an amount, concentration, or other quantitative or qualitative assessment is made upon either one or more RNA species or total extracellular RNA without amplification prior to detection. For example but not limitation, an extracellular RNA species extracted from a bodily fluid may be hybridized and detected without amplification. In this aspect, it is particularly preferred but not required that the extracted RNA be concentrated upon extraction or upon separation from the bodily fluid, using for example immunobead capture or hybridization onto a solid substrate, to improve assay sensitivity, or by centrifugation of the non-cellular bodily fluid at centrifugation speeds high enough to concentrate apoptotic bodies and particles, and extracellular RNA thereby, in the pellet, upon which RNA extraction and hybridization may be performed. In another aspect of this embodiment, extracellular RNA or total RNA is evaluated by spectroscopy, for example by mass spectroscopy or magnetic resonance spectroscopy, or by flow cytometry, thereupon a determination can be made relative to an extracellular RNA species. In one aspect, fluorometric methods may be employed, for example as employed by Kamm and Smith (1972, Clin. Chem. 18: 519-522), said reference incorporated herein in its entirety, whereupon a determination is then made relative to an extracellular RNA species.

The methods of the invention identify humans or animals bearing or at risk for developing malignancies including but not limited to tumors of breast, ovarian, lung, cervical, colorectal, gastric, pancreatic, bladder, endometrial, head and neck, brain, kidney, and esophageal tissues, as well as leukemias, lymphomas, melanoma, and sarcomas. The methods of the invention may further be utilized to identify humans or animals with premalignancy, including but not limited to colorectal adenoma, cervical dysplasia, cervical intraepithelial neoplasia (CIN), bronchial dysplasia, bronchial metaplasia, atypical hyperplasia of the breast, ductal carcinoma in-situ of the breast, atypical endometrial hyperplasia, prostatic intraepithelial neoplasia, and Barrett's esophagus. The methods of the invention may be applied to a subject of any age, race, ethnicity or gender, although it is preferred that the reference group or population include individuals of similar age group (for example, child, adult, elderly or over 65 years old) and sex (male, female).

The invention permits detection, diagnosis, evaluation, characterization and monitoring of disease, particularly cancer and premalignancy, and identification of individuals at risk for developing disease, particularly cancer or neoplastic disease such as premalignancy, providing considerable clinical utility. The invention provides methods to identify, stratify, or select a human or animal that might benefit from a therapy, or from a further diagnostic test. The invention permits disease such as cancer to be monitored, including response to therapies such as cancer therapies, by providing a biomarker to guide whether therapeutic effect has been achieved, or if more therapy is required, and to assess prognosis.

An advantageous application of the methods of this invention is to allow selection of humans or animals for cancer therapies including surgery, biotherapy, hormonal therapy, anti-sense therapy, monoclonal antibody therapy, chemotherapy, vaccines, anti-angiogenic therapy, cryotherapy, radiation therapy, and RNA inhibitor-directed therapies, including therapies interfering with RNA or DNA expression and including miRNA-directed therapies and si-RNA-directed therapies, adjuvant therapies, neoadjuvant therapies, and combined modality therapies.

Another advantageous application of the methods of this invention is to provide an indicator of a relapsed cancer following therapy, or impending relapse, or treatment failure.

Another advantageous application of the methods of this invention is to identify humans or animals who might benefit from additional diagnostic procedures, wherein said procedures include but are not limited to surgery, biopsy, needle aspiration, radiological imaging including X-ray, MRI, and CT scanning, radionucleotide imaging, colonoscopy, sigmoidoscopy, bronchoscopy, endoscopy, PET scanning, stool analysis, sputum analysis, cystoscopy, pelvic examination including PAP, and physical examination.

The invention further provides kits for research, diagnostic, or other clinical use that enable quantitative, qualitative assessment of total extracellular RNA or of specific RNA species in plasma, serum, or other non-cellular bodily fluids. In one aspect, a kit according to this aspect of the invention can provide a reference range for normal values or values that are disease-related under conditions that enable identification or selection of a human or animal with a disease, most particularly cancer or neoplastic disease. In another aspect kits of this invention comprise one or more of the following: reagents for extracting total RNA from plasma, serum, or other bodily fluid; a stabilizing agent or reagents; reverse transcriptase; reagents and/or probes and primers for the quantitative or quantitative amplification or signal amplification of said RNA or cDNA derived therefrom; reagents and materials for the detection of RNA or cDNA product; reagents for hybridization of RNA or cDNA; standards and controls for the analysis of the test including a negative control and/or RNA or cDNA positive control or RNA or cDNA standard; reagents or devices or tubes for collecting, handling, or storage of the plasma, serum, or bodily fluid; solid substrate platforms for detection of RNA or cDNA therefrom or their amplified or signal amplified product; or any combination or variation thereof, wherein further the reagents or kit components may be standardized to be comparable with reagents used to define RNA values for the reference population.

The methods of the invention and preferred uses for the methods of the invention are more fully illustrated in the following Examples. These Examples illustrates certain aspects of the above-described method and advantageous results. These Examples are shown by way of illustration and not by way of limitation.

### EXAMPLE 1

Quantitative analysis of two or more extracellular RNA species, and application of a stabilizing agent:

Quantitative analysis for two tumor-related mRNA, Her-2/neu RNA and hTERT RNA, in plasma is evaluated in a human. Blood is drawn in a local laboratory facility using an EDTA containing vacutainer tube from a 48 year old woman with a suspicious breast mass. Within 3 hours following venipuncture, the blood specimen is centrifuged at 3000×g for 15 minutes to isolate a non-cellular bodily fluid fraction comprising blood plasma, and then Trizol 2 milliliters is added to 2 milliliters of plasma. The plasma specimen mixed with Trizol is then shipped to a central laboratory. Total RNA is extracted from plasma according to manufacturer's instructions using Trizol. The extracted RNA from 50 microliters of plasma is then reverse transcribed and Her-2/neu cDNA and hTERT cDNA amplified quantitatively using Taqman technology and using labeled cDNA-specific primers. The amount or concentration of each RNA species is comparatively analyzed, thereby supporting a diagnosis of breast cancer, wherein elevation of both Her-2/neu RNA and hTERT RNA quantitative levels to an extent twice greater than found in a normal reference standard is assigned with a known specificity to be considered consistent with a malignancy being present, and further consistent with breast cancer. The assay is repeated serially throughout treatment thereby enabling the characterization of the cancer and monitoring response to therapy. The cancer is characterized as a Her-2/neu overexpressing tumor and the patient can be treated with Herceptin and monitored. It is understood that additional extracellular RNA species could be similarly evaluated, and other tumor types characterized.

### EXAMPLE 2

Quantitative analysis of two or more extracellular RNA in addition to extracellular DNA in the absence of stabilizing agent: A 62 year old man with a long-time history of ulcerative colitis is screened for colorectal cancer. A blood specimen is obtained in an EDTA-containing blood tube. The plasma non-cellular bodily fluid fraction is isolated from the cellular blood by centrifuging the tube at 1500×g for 15 minutes. Total RNA and DNA is then extracted from the plasma and reverse transcribed, thereby reverse transcribing EGFr RNA, c-abl RNA, and hTERT RNA to their respective cDNA. The cDNA product and mutant K-ras DNA is then quantitatively amplified by PCR in a multiplexed fashion using primers specific for mutant K-ras DNA, c-abl cDNA, EGFr cDNA, and hTERT cDNA, and the amplified products then quantitatively detected. The c-abl cDNA product value is quantitatively within the range expected for a c-abl cDNA normal reference value, and the mutant K-ras DNA, EGFr cDNA, and hTERT cDNA product values are all above the range expected within normal reference values and greater than the specimen c-abl RNA and c-abl RNA reference values. The degree of elevated value and pattern of positivity thereby enables detection and characterization of colorectal cancer in the patient. The RNA signature profile further supports a determination of malignancy in this patient, where the profile is assessed relative to normal and cancer reference profiles.

### EXAMPLE 3

Clinical Application

A 52 year-old woman with no symptomatic evidence of disease presents for routine cancer screening. Her physician draws an anticoagulated blood specimen for assay, wherefrom blood plasma is isolated by centrifugation. Total extracellular RNA is extracted from the patient's plasma, and the extracted extracellular RNA amplified quantitatively using Taqman PCR technology for an RNA species encoding a housekeeping gene or similar standard RNA such as c-abl RNA, and for a tumor-associated RNA such as EGFr RNA. In this case the woman's quantitative levels of EGFr RNA are substantially elevated relative to an EGFr RNA reference standard for healthy patients without cancer, while levels of c-abl RNA are consistent with the normal reference range of c-abl RNA in plasma from healthy patients without cancer. The difference between the two ratios indicates that either the presence of cancer, or a high risk of developing cancer, is therefore identified for the woman.

### EXAMPLE 4

Clinical Application:

A 49 year old man receives colorectal cancer screening after his brother was recently diagnosed with colon cancer. The man provides a saliva sample to his physician. The saliva is isolated, preferably by suctioning about 1 or more ml volume of saliva from the man's mouth, or by having the man spit into a container. Total RNA, and further extracellular DNA, is then extracted directly from the saliva using methods as described herein, and the extracted RNA is reverse transcribed, and amplified or signal amplified in a qualitative or quantitative fashion for cDNA corresponding to a group of RNA species, and further DNA species, that characterizes between patients with colorectal cancer and healthy individuals. The group of RNA species and DNA species can include, but is not limited to, hTERT RNA, hTR RNA, EGFr mRNA, COX-2 mRNA, mutated K-ras DNA, APC DNA, and in addition housekeeper gene RNA such as c-abl RNA. Detection of extracellular RNA and extracellular DNA species in the predominantly acellular saliva specimen enables a determination of an extracellular nucleic acid pattern that in this patienti consistent with the presence of either colorectal cancer or colorectal adenoma. The man is then referred for colonoscopy (a more invasive and costly procedure) for further evaluation and diagnosis. Since in this example the detected tumor-associated RNA and DNA species are from a neoplasm that is not contiguous with the oral cavity, the RNA and DNA species would be extracellular species, and be unlikely to reflect cellular contamination.

It should be understood that the foregoing disclosure emphasizes certain specific embodiments of the invention and that all modifications or alternatives equivalent thereto are within the spirit and scope of the invention as set forth in the appended claims.

What is claimed is:

1. A method of diagnosing, detecting, evaluating, characterizing, or monitoring cancer or other disease in a human or animal, the method comprising the steps of:
a) isolating a non-cellular bodily fluid fraction from a bodily fluid of a human or animal;
b) extracting RNA from the non-cellular bodily fluid fraction, wherein said extracted RNA comprises two or more RNA species;
c) quantitatively amplifying or signal amplifying two or more RNA species, or cDNA therefrom, comprising a portion of the extracted RNA, to provide quantitative amounts or concentrations of said RNA species;
d) detecting quantitative amounts or concentrations of the amplified or signal amplified RNA or cDNA products; and
e) diagnosing, detecting, evaluating, characterizing, or monitoring a cancer or other disease thereby.

2. The method of claim 1, wherein the non-cellular bodily fluid fraction is blood plasma or serum.

3. The method of claim 1, wherein a non-cellular bodily fluid fraction is isolated from a bodily fluid by centrifugation.

4. The method of claim 1, wherein a non-cellular bodily fluid fraction is isolated from a bodily fluid by size fractionation.

5. The method of claim 1, wherein a non-cellular bodily fluid fraction is isolated from a bodily fluid by passing a bodily fluid through a filter.

6. The method of claim 1, wherein at least one of the RNA species is a tumor-associated or tumor-derived RNA.

7. The method of claim 1, wherein the amount or concentration of at least one of the RNA species in the non-cellular bodily fluid is greater than the amount or concentration of said RNA species in humans without cancer.

8. The method of claim 1, wherein the amount or concentration of a least one of the RNA species in the non-cellular bodily fluid is the amount or concentration of said RNA species in humans with cancer.

9. A method of determining the relative quantitative amounts or concentrations of two or more extracellular RNA in a bodily fluid of a human or animal, the method comprising the steps of:
a) isolating a non-cellular bodily fluid fraction from a bodily fluid of a human or animal;
b) extracting RNA from the non-cellular bodily fluid fraction;
c) quantitatively amplifying or signal amplifying two or more extracellular RNA species or cDNA therefrom, comprising a portion of the extracted RNA, to provide quantitative amounts or concentrations of said extracellular RNA species or cDNA therefrom; and
d) determining the relative amounts or concentrations of two or more extracellular RNA in a bodily fluid of a human or animal thereby.

10. The method of claim 9, whereby a disease is thereby detected, diagnosed, characterized, evaluated, or monitored in the human or animal.

11. The method of claim 9, wherein the non-cellular bodily fluid fraction is blood plasma or serum.

12. The method of claim 9, wherein the bodily fluid is blood.

13. The method of claim 9, wherein a non-cellular bodily fluid fraction is isolated from a bodily fluid by centrifugation.

14. The method of claim 9, wherein a non-cellular bodily fluid fraction is isolated from a bodily fluid by size fractionation.

15. The method of claim 9, wherein a non-cellular bodily fluid is isolated from a bodily fluid by passing a bodily fluid through a filter.

16. The method of claim 9, wherein at least one of the RNA species is a tumor-associated or tumor-derived RNA.

17. The method of claim 9, wherein at least one of the RNA species is a non-tumor RNA.

18. The method of claim 10, wherein the disease is cancer.

19. A kit for performing the method of claim 1, comprising one or more of the following:
a) reagents for extracting RNA from plasma or serum or other non-cellular bodily fluid;
b) reagents for amplification or signal amplification;
c) reagents for hybridization of RNA or cDNA;
d) probes or primers for amplification or signal amplification of RNA or cDNA derived therefrom;
e) RNA or cDNA standards or controls;
f) a negative control;
g) reverse transcriptase;
h) reagents or materials for the detection of amplified or signal amplified RNA or cDNA product;
i) reagents or devices for the collecting, handling, or storage of bodily fluid;
j) solid substrate platforms for the detection of RNA or cDNA derived therefrom or of RNA or cDNA amplified or signal amplified product;
k) a set of reference values, standards, or specimens; and
l) any combination or variation thereof.

20. A kit for performing the method of claim 9, comprising one or more of the following:
a) reagents for extracting RNA from plasma or serum or other non-cellular bodily fluid;
b) reagents for amplification or signal amplification;
c) reagents for hybridization of RNA or cDNA;
d) probes or primers for amplification or signal amplification of RNA or cDNA derived therefrom;
e) RNA or cDNA standards or controls;
f) a negative control;
g) reverse transcriptase;
h) reagents or materials for the detection of amplified or signal amplified RNA or cDNA product;
i) reagents or devices for the collecting, handling, or storage of bodily fluid;
j) solid substrate platforms for the detection of RNA or cDNA derived therefrom or of RNA or cDNA amplified or signal amplified product;
k) a set of reference values, standards, or specimens; and
l) any combination or variation thereof.
